(19) United States
(12) Patent Application Publication
(10) Pub. No.: US 2026/0108477 A1
(43) Pub. Date: Apr. 23, 2026

(54) CYCLOBENZAPRINE TREATMENT FOR FIBROMYALGIA

(71) Applicant: TONIX PHARMA LIMITED, DUBLIN (IE)

(72) Inventors: Seth Lederman, Austin, TX (US); Gregory M. Sullivan, Barryville, NY (US)

(21) Appl. No.: 19/421,039

(22) Filed: Dec. 16, 2025

Related U.S. Application Data
(63) Continuation of application No. 18/265,525.
(60) Provisional application No. 63/122,469, filed on Dec. 7, 2020.

(51) Int. Cl. A61K 31/135; A61K 9/00; A61P 25/00

(52) U.S. Cl. CPC A61K 31/135; A61K 9/006; A61P 25/00

(57) ABSTRACT

Methods for treating fibromyalgia and one or more of its associated symptoms of pain, sleep disturbance and/or fatigue comprising administering to a subject in need thereof, 5.6 mg cyclobenzaprine HCl per day in one or more dosage units by transmucosal administration, the cyclobenzaprine HCl being in a form of a eutectic, and the one or more dosage units further comprising a basifying agent.

### BACKGROUND

Cyclobenzaprine, or 3-(5H-dibenzola[a,d]cyclohepten-5-ylidene)-N,N-dimethyl-1 propanamine, was first approved by the U.S. Food and Drug Administration in 1977 for the treatment of acute muscle spasms of local origin. (Katz and Dube, 1988).

An estimated 6-12 million adults in the United States have fibromyalgia and 90% of whom are women. Fibromyalgia is considered a central nervous system disorder with symptoms including chronic widespread pain, nonrestorative sleep, fatigue, diminished cognition and mood disturbances. These symptoms are believed to result from inappropriate pain signaling in the central nervous system in the absence of peripheral injury. Fibromyalgia causes significant impairment in all areas in life where patients present with lower levels of health-related quality of life, such as reduced daily functioning and interference with work (e.g., loss of productivity and disability). There is an unmet need to treat fibromyalgia as fewer than half of the fibromyalgia patients can receive complete relief from the current three FDA-approved drugs. Moreover, there is substantial off-label use of narcotic painkillers and prescription sleep aids. Among those diagnosed, more than one-third have used prescription opioids as a means of treatment. Cyclobenzaprine HCl, as described in various embodiments of this disclosure meets this unmet need and improves pain, sleep quality and fatigue for subjects suffering from fibromyalgia.

### SUMMARY OF THIS DISCLOSURE

One aspect of this disclosure provides a method of treating fibromyalgia and inter alia one or more of its associated symptoms of pain, sleep disturbance and/or fatigue, comprising administering to a subject in need thereof, 5.6 mg cyclobenzaprine HCl per day in one or more dosage units by transmucosal administration, the cyclobenzaprine HCl being in a form of a eutectic comprising 75%±2% cyclobenzaprine HCl and 25%±2% mannitol, and the one or more dosage units further comprising a basifying agent.

Another aspect of this disclosure provides a method of treating fibromyalgia and its associated symptoms of pain, sleep disturbance and/or fatigue, comprising administering to a subject in need thereof, 5.6 mg cyclobenzaprine HCl per day in one or more dosage units by transmucosal administration, the cyclobenzaprine HCl being in a form of a eutectic comprising 75%±2% cyclobenzaprine HCl and 25%±2% mannitol, and the one or more dosage units further comprising a basifying agent.

In some embodiments of this disclosure the cyclobenzaprine HCl eutectic may instead be selected from the group consisting of a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol. It should be understood that the “cyclobenzaprine HCl eutectic” of this disclosure refers to any of these eutectics or granules.

In some embodiments of this disclosure, the one or more dosage units comprising the cyclobenzaprine HCl eutectic are two dosage units, each dosage unit comprising 2.8 mg of cyclobenzaprine HCl.

Another aspect of this disclosure provides a multiple-variable dose method of treating fibromyalgia and inter alia one or more of its associated symptoms of pain, sleep disturbance and/or fatigue in a subject in need thereof, comprising the transmucosal administration of:
one or more of a first dosage unit comprising 2.8 mg cyclobenzaprine HCl the subject daily for about two weeks; and
one or more of a second dosage unit comprising 5.6 mg of cyclobenzaprine HCl to the subject daily for as long as needed,
wherein the cyclobenzaprine HCl is in the form of a eutectic, and
wherein the one of more dosage units further comprise a basifying agent.

Another aspect of this disclosure provides a multiple-variable dose method of treating fibromyalgia and its associated symptoms of pain, sleep disturbance and/or fatigue in a subject in need thereof, comprising the transmucosal administration of:
one or more of a first dosage unit comprising 2.8 mg cyclobenzaprine HCl to the subject daily for about two weeks; and
one or more of a second dosage unit comprising 5.6 mg of cyclobenzaprine HCl to the subject daily for as long as needed, the one or more second dosage unit being administered following the two-week administration of the one or more first dosage unit,
wherein the cyclobenzaprine HCl is in the form of a eutectic, and
wherein the one of more dosage units further comprise a basifying agent.

Another aspect of this disclosure provides a multiple-variable dose method of treating fibromyalgia and its associated symptoms of pain, sleep disturbance and/or fatigue in a subject in need thereof, comprising the transmucosal administration of:
one or more of a first dosage unit comprising 2.8 mg cyclobenzaprine HCl to the subject daily for about two weeks; and
one or more of a second dosage unit comprising 5.6 mg of cyclobenzaprine HCl to the subject daily for as long as needed, the one or more second dosage unit being administered following the two-week administration of the one or more first dosage unit,
the cyclobenzaprine HCl in each of the one or more dosage units being in the form of a eutectic comprising 75%±2% cyclobenzaprine and 25%±2% mannitol, and each of the one or more dosage units further comprising a basifying agent.

In some embodiments of this disclosure the cyclobenzaprine HCl eutectic of the first dosage unit may instead be selected from the group consisting of a 65%±2% cyclobenzaprine HCl and 35%+2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol.

In some embodiments of this disclosure the cyclobenzaprine HCl eutectic of the second dosage unit may instead be selected from the group consisting of a 65%±2% cyclobenzaprine HCl and 35%±2% δ-mannitol eutectic, a mixture of a 75%±2% cyclobenzaprine HCl and 25%+2% β-mannitol and a 65%+2% cyclobenzaprine HCl and 35%±2% δ-mannitol eutectic, and a granule comprising an outer layer of a 65%±2% cyclobenzaprine HCl and 35%+2% δ-mannitol eutectic and an inner layer of β-mannitol.

In some embodiments, one or more the dosage units comprising the cyclobenzaprine HCl eutectic are administered daily at bedtime.

In some embodiments, all of the dosage units of the cyclobenzaprine HCl eutectic are administered daily at bedtime.

In some embodiments, the transmucosal administration comprises sublingual, buccal, intranasal or palatal.

In some embodiments, the transmucosal administration is sublingual.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, conjugate bases of some organic acids, bicarbonate, and sulfide.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

In some embodiments, the basifying agent is dipotassium hydrogen phosphate.

In some embodiments, the mannitol is β-mannitol or δ-mannitol.

In some embodiments, the mannitol is β-mannitol.

In some embodiments, the mannitol is δ-mannitol.

In some embodiments, the method of treatment reduces pain.

In some embodiments, the pain is measured by daily diary pain severity score change from a baseline score as compared to a placebo group using a numerical rating scale.

In some embodiments, the pain is reduced by greater than 30%.

In some embodiments, the method of treatment improves sleep quality or reduces sleep disturbances.

In some embodiments, method of treatment reduces fatigue.

In some embodiments, the subject is human.

Another aspect of this disclosure provides a composition for use in treating fibromyalgia and inter alia one or more of its associated symptoms of pain, sleep disturbance and/or fatigue, the composition being suitable for transmucosal administration and comprising 5.6 mg cyclobenzaprine HCl in one or more dosage units, the cyclobenzaprine HCl being in the form of a eutectic comprising a 75%±2% cyclobenzaprine HCl and 25%±2% mannitol eutectic, a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, or a granule comprising an outer layer of a 65%±2% cyclobenzaprine HCl and 35%±2% δ-mannitol eutectic and an inner layer of β-mannitol, and the one or more dosage units further comprising a basifying agent.

Another aspect of this disclosure provides a composition for use in treating fibromyalgia and its associated symptoms of pain, sleep disturbance and/or fatigue, the composition being suitable for transmucosal administration and comprising 5.6 mg cyclobenzaprine HCl in one or more dosage units, the cyclobenzaprine HCl being in the form of a eutectic comprising 75%±2% cyclobenzaprine HCl and 25%±2% mannitol, and the one or more dosage units further comprising a basifying agent.

In some embodiments of this disclosure, the cyclobenzaprine HCl eutectic in the composition for use may instead be selected from the group consisting of a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%+2% δ-mannitol eutectic and an inner layer of β-mannitol.

In some embodiments, the cyclobenzaprine HCl eutectic in the composition for use comprises a 75%±2% cyclobenzaprine and 25%±2% mannitol eutectic.

In some embodiments, the composition of this disclosure is administered in two dosage units, each dosage unit comprising 2.8 mg of cyclobenzaprine HCl in the form of a eutectic.

In some embodiments, the composition is administered at bedtime.

In some embodiments, the transmucosal administration comprises sublingual, buccal, intranasal or palatal.

In some embodiments, the transmucosal administration is sublingual.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, conjugate bases of some organic acids, bicarbonate, and sulfide.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

In some embodiments, the basifying agent is potassium phosphate dibasic.

In some embodiments, the mannitol is β-mannitol or δ-mannitol.

In some embodiments, the mannitol is β-mannitol.

In some embodiments, the mannitol is δ-mannitol.

In some embodiments, the associated symptom is pain.

In some embodiments, the pain is measured by daily diary pain severity score change from a baseline score as compared to a placebo group using a numerical rating scale.

In some embodiments, the pain is reduced by greater than 30%.

In some embodiments, the associated symptom is sleep disturbances.

In some embodiments, the associated symptom is fatigue.

### BRIEF DESCRIPTION OF DRAWINGS

FIG. 1 shows a graph of least squares mean change from baseline in Numeric Rating Scale (NRS) pain score. Pain score was measured and compared between Placebo (N=255) and Cyclobenzaprine HCl (N=248) groups once a week for 14 weeks.

FIG. 2 shows a Continuous Responder Analysis (CRA) graph. The proportion of responders in Placebo and Cyclobenzaprine HCl groups over an entire-range of cut-off points (e.g., 0% to 100%) for percentage pain responders.

### DETAILED DESCRIPTION

The present disclosure provides in some embodiments methods and compositions for treating fibromyalgia and its associated symptoms of pain, sleep disturbance and fatigue in a subject in need thereof, the composition being suitable for once a day transmucosal administration and comprising 5.6 mg cyclobenzaprine HCl in one or more dosage units, the cyclobenzaprine HCl being in the form of a eutectic comprising 75%±2% cyclobenzaprine HCl and 25%±2% mannitol, and the one or more dosage units further comprising a basifying agent.

### Definitions

The term “herein” means the entire application.

Unless otherwise defined herein, scientific and technical terms used in this application shall have the meanings that are commonly understood by those of ordinary skill in the art. In case of conflict, the present specification, including definitions, will control.

It should be understood that any of the embodiments described herein, including those described under different aspects of the disclosure and different parts of the specification (including embodiments described only in the Examples) can be combined with one or more other embodiments of this disclosure, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, the term “about” refers to a value or parameter that includes (and describes) embodiments that are directed to that value or parameter per se. For example, description referring to “about X” includes description of “X.” As used herein, the term “about” permits a variation off ±10% within the range of the significant digit.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments. The materials, methods, and examples are illustrative only and not intended to be limiting.

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “treat” and its cognates refer to a full or partial amelioration or modulation of fibromyalgia or at least one discernible symptom therein. In some embodiments, “treat at least one discernible symptom” refers to a reduction of pain. In some embodiments, “treat” refers to an improvement of pain score, i.e. a reduction in pain, as an associated symptom of fibromyalgia. In some embodiments, “treat of at least one discernible symptom” refers to reduction of sleep disturbance. In some embodiments, “treat” refers to an improvement in sleep quality. In some embodiments, “treat at least one discernible symptom” refers to a reduction of fatigue. In some embodiments, “treat” refers to “much improved” or “very much improved” in the context of these associated symptoms.

In the embodiments of this disclosure, the cyclobenzaprine HCl eutectic is administered together with a basifying agent. See, e.g., WO2013/188847, incorporated herein by reference.

The “basifying agent” included in some embodiments of this disclosure is selected from a group consisting of potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide. In some embodiments, the basifying agent is potassium dihydrogen phosphate (monopotassium phosphate, monobasic potassium phosphate, KH₂PO₄) or dipotassium hydrogen phosphate (dipotassium phosphate, dibasic potassium phosphate, K₂HPO₄). In some embodiments, the basifying agent is an ingredient (and excipient) in a tablet, and the basifying agent exerts its effects during the time the tablet is being dispersed in the mucous material, while parts of the formulation are dissolving in the mucous material and for a period of time after the tablet is dissolved in the mucous material. A basifying agent with particular effects on cyclobenzaprine HCl is dipotassium hydrogen phosphate (K₂HPO₄). Another basifying agent with particular effects on cyclobenzaprine HCl is potassium dihydrogen phosphate (KH₂PO₄). Another basifying agent with particular effects on cyclobenzaprine HCl is disodium hydrogen phosphate (Na₂HPO₄). Another basifying agent with particular effects on cyclobenzaprine HCl is tripotassium citrate. Another basifying agent with particular effects on cyclobenzaprine HCl is trisodium citrate.

In some embodiments the cyclobenzaprine HCl eutectic of this disclosure is selected from the group consisting of the one of the eutectics or granules referred to in paragraph [0005] above. In some embodiments of this disclosure, the cyclobenzaprine HCl is in the form of a 75%±2% cyclobenzaprine HCl and 25%±2% mannitol eutectic. In some embodiments of this disclosure, the cyclobenzaprine HCl is in the form of a 75%±2% cyclobenzaprine HCl and 25%±2% β-mannitol eutectic. See, e.g., WO2014/145156, incorporated herein by reference.

As used herein, the term a “eutectic” or “in the form of a eutectic” refers to a mixture of chemical compounds or elements that has a single chemical composition that melts at a lower temperature than any other composition made up of the same ingredients. A composition comprising a eutectic is known as the eutectic composition and its melting temperature is known as the eutectic temperature. Eutectic compositions often have higher stability and/or dissolution rates than their non-eutectic counterparts. Because eutectics enhance dissolution, they can be employed to increase permeability in solid dispersions and dispersion systems.

Any suitable transmucosal route of administration may be employed for providing the subject with the dosage units of this disclosure. For example, transmucosal administration including sublingual, buccal, intranasal, palatal and the like may be employed as appropriate. In some embodiments, the dosage form is a sublingual tablet, a sublingual film, a liquid, sublingual powder, or a sublingual spray solution.

### Methods for Treating

In some embodiments, the present disclosure provides a method for treating fibromyalgia and its associated symptoms of pain, sleep disturbance and/or fatigue, comprising administering to a subject in need thereof, 5.6 mg cyclobenzaprine HCl per day in one or more dosage units by transmucosal administration, the cyclobenzaprine HCl being in a form of a eutectic comprising 75%±2% cyclobenzaprine HCl and 25%±2% mannitol, and the one or more dosage units further comprising a basifying agent. In some embodiments the cyclobenzaprine HCl eutectic is a 75%±2% cyclobenzaprine HCl and 25%±2% β-mannitol eutectic.

In some other embodiments, the cyclobenzaprine HCl is in a form of a eutectic selected from the group consisting of a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol. In some embodiments, the eutectic is a 65%±2% cyclobenzaprine HCl and 35%+2% δ-mannitol eutectic. In some embodiments, the cyclobenzaprine HCl eutectic is a mixture of a 75%±2% cyclobenzaprine HCl and 25%±2% β-mannitol eutectic and a 65%±2% cyclobenzaprine HCl and 35%±2% δ-mannitol eutectic. In some embodiments, the cyclobenzaprine HCl eutectic is a granule comprising an outer layer of 65%±2% cyclobenzaprine HCl and 35%±2% δ-mannitol eutectic and an inner layer of β-mannitol.

In some embodiments, the cyclobenzaprine HCl eutectic is administered as one or more dosage units. In some embodiments, the cyclobenzaprine HCl eutectic is administered in two dosage units. In some embodiments, the cyclobenzaprine HCl eutectic is administered in two dosage units, each dosage unit comprising 2.8 mg of cyclobenzaprine HC.

Another aspect, disclosed herein is a multiple-variable dose method of treating fibromyalgia and inter alia one or more of its associated symptoms of pain, sleep disturbance and/or fatigue in a subject in need thereof, comprising the transmucosal administration of:
2.8 mg cyclobenzaprine HCl in one or more of a first dosage unit to the subject daily for about two weeks; and
5.6 mg of cyclobenzaprine HCl in one or more of a second dosage unit administered to the subject daily for as long as needed,
wherein the cyclobenzaprine HCl is in the form of a eutectic, and
wherein the one of more dosage units further comprise a basifying agent.

Another aspect, disclosed herein is a multiple-variable dose method of treating fibromyalgia and its associated symptoms of pain, sleep disturbance and/or fatigue in a subject in need thereof, comprising the transmucosal administration of:
2.8 mg cyclobenzaprine HCl in one or more of a first dosage unit to the subject daily for about two weeks; and
5.6 mg of cyclobenzaprine HCl in one or more of a second dosage unit administered to the subject daily for as long as needed, the one or more second dosage unit being administered following the two-week administration of the one or more first dosage unit,
wherein the cyclobenzaprine HCl is in the form of a eutectic, and
wherein the one of more dosage units further comprise a basifying agent.

In some embodiments, the cyclobenzaprine HCl of the first dosage unit is in the form of a eutectic comprising 75%±2% cyclobenzaprine and 25%±2% mannitol. In some embodiments, the cyclobenzaprine HCl of the second dosage unit is in the form of a eutectic comprising 75%±2% cyclobenzaprine and 25%±2% mannitol.

In some embodiments of this disclosure the cyclobenzaprine HCl eutectic of the first dosage unit may instead be selected from the group consisting of a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%±2% δ-mannitol eutectic and an inner layer of β-mannitol. In some embodiments of this disclosure the cyclobenzaprine HCl eutectic of the first dosage unit is a 65%±2% cyclobenzaprine HCl and 35%±2% δ-mannitol eutectic. In some embodiments of this disclosure the cyclobenzaprine HCl eutectic of the first dosage unit is a mixture of a 75%±2% cyclobenzaprine HCl and 25%±2% β-mannitol and a 65%±2% cyclobenzaprine HCl and 35%±2% δ-mannitol eutectic. In some embodiments of this disclosure the cyclobenzaprine HCl eutectic of the first dosage unit is a granule comprising an outer layer of a 65%±2% cyclobenzaprine HCl and 35%±2% δ-mannitol eutectic and an inner layer of β-mannitol.

In some embodiments of this disclosure the cyclobenzaprine HCl eutectic of the second dosage unit may instead be selected from the group consisting of a 65%±2% cyclobenzaprine HCl and 35%±2% δ-mannitol eutectic, a mixture of a 75%±2% cyclobenzaprine HCl and 25%±2% β-mannitol and a 65%±2% cyclobenzaprine HCl and 35%±2% δ-mannitol eutectic, and a granule comprising an outer layer of a 65%±2% cyclobenzaprine HCl and 35%+2% δ-mannitol eutectic and an inner layer of β-mannitol. In some embodiments of this disclosure the cyclobenzaprine HCl eutectic of the second dosage unit is a 65%±2% cyclobenzaprine HCl and 35%±2% δ-mannitol eutectic. In some embodiments of this disclosure the cyclobenzaprine HCl eutectic of the second dosage unit is a mixture of a 75%±2% cyclobenzaprine HCl and 25%±2% β-mannitol and a 65%±2% cyclobenzaprine HCl and 35%+2% δ-mannitol eutectic. In some embodiments of this disclosure the cyclobenzaprine HCl eutectic of the second dosage unit is a granule comprising an outer layer of a 65%±2% cyclobenzaprine HCl and 35%±2% δ-mannitol eutectic and an inner layer of β-mannitol.

Another aspect, disclosed herein is a multiple-variable dose method of treating fibromyalgia and its associated symptoms of pain, sleep disturbance and/or fatigue in a subject in need thereof, comprising the transmucosal administration of:
2.8 mg cyclobenzaprine HCl in one or more of a first dosage unit to the subject daily for about two weeks; and
5.6 mg of cyclobenzaprine HCl in one or more of a second dosage unit administered to the subject daily for as long as needed, the one or more second dosage unit being administered following the two-week administration of the one or more first dosage unit, the cyclobenzaprine HCl in each of the one or more dosage units being in the form of a eutectic comprising 75%±2% cyclobenzaprine HCl and 25%±2% mannitol, and each of the one or more dosage units further comprising a basifying agent.

In some embodiments, the one or more second dosage unit is administered following administration of the one or more first dosage unit.

In some embodiments, the one or more of the dosage units comprising the cyclobenzaprine HCl eutectic are administered at bedtime. In some embodiments, all of the dosage units comprising the cyclobenzaprine HCl eutectic are administered daily at bedtime.

In some embodiments, transmucosal administration comprises sublingual, buccal, intranasal or palatal. In some embodiments, transmucosal administration comprises sublingual. In some embodiments, transmucosal administration comprises buccal. In some embodiments, transmucosal administration comprises intranasal. In some embodiments, transmucosal administration comprises palatal.

In some embodiments, the dosage form is a sublingual tablet, a sublingual film, a liquid, sublingual powder, or a sublingual spray solution. In some embodiments, the dosage form is a sublingual tablet. In some embodiments, the dosage form is a sublingual film. In some embodiments, the dosage form is a liquid. In some embodiments, the dosage form is sublingual powder. In some embodiments, the dosage form is a sublingual spray solution.

In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, the conjugate bases of some organic acids (including bicarbonate), and sulfide. In some embodiments, the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate. In some embodiments, the basifying agent is potassium dihydrogen phosphate. In some embodiments, the basifying agent is dipotassium hydrogen phosphate. In some embodiments, the basifying agent is tripotassium phosphate. In some embodiments, the basifying agent is sodium carbonate. In some embodiments, the basifying agent is sodium bicarbonate. some embodiments, the basifying agent is calcium carbonate. In some embodiments, the basifying agent is calcium bicarbonate. In some embodiments, the basifying agent is TRIS buffer. In some embodiments, the basifying agent is sodium dihydrogen phosphate. In some embodiments, the basifying agent is disodium hydrogen phosphate. In some embodiments, the basifying agent is trisodium phosphate. In some embodiments, the basifying agent is potassium carbonate. In some embodiments, the basifying agent is potassium bicarbonate. In some embodiments, the basifying agent is potassium acetate. In some embodiments, the basifying agent is sodium acetate. In some embodiments, the basifying agent is dipotassium citrate. In some embodiments, the basifying agent is tripotassium citrate. In some embodiments, the basifying agent is disodium citrate and trisodium citrate.

In some embodiments, the mannitol is β-mannitol or δ-mannitol. In some embodiments, the mannitol is β-mannitol. In some embodiments, the mannitol is δ-mannitol.

In some embodiments, the treatment reduces the associated symptom of pain. In some embodiments, the treatment significantly reduces pain. In some embodiments, pain is measured by daily diary pain severity score change from a baseline score as compared to a placebo group using a numerical rating scale. In some embodiments, pain is reduced by greater than 30%.

In some embodiments, the treatment improves the associated symptom of sleep quality or reduces sleep disturbances. In some embodiments, the treatment significantly improves sleep quality or reduces sleep disturbances.

In some embodiments, the treatment reduces the associated symptom of fatigue. In some embodiments, the treatment significantly reduces fatigue.

In another aspect, disclosed herein is a composition for use in treating a subject believed to have fibromyalgia and its associated symptoms of pain, sleep disturbance and/or fatigue, the composition being suitable for transmucosal administration and comprising 5.6 mg cyclobenzaprine HCl in one or more dosage units, the cyclobenzaprine HCl being in the form of a eutectic comprising 75%±2% cyclobenzaprine HCl and 25%±2% mannitol, and the one or more dosage units further comprising a basifying agent.

In another aspect, disclosed herein is a composition for use in treating a subject believed to have fibromyalgia and inter alia one or more of its associated symptoms of pain, sleep disturbance and/or fatigue, the composition being suitable for transmucosal administration and comprising 5.6 mg cyclobenzaprine HCl in one or more dosage units, the cyclobenzaprine HCl being in the form of a eutectic comprising 75%±2% cyclobenzaprine HCl and 25%±2% β-mannitol, and the one or more dosage units further comprising a basifying agent.

Another aspect of this disclosure is a composition for use in treating a subject believed to have fibromyalgia and its associated symptoms of pain, sleep disturbance and/or fatigue, the composition being suitable for transmucosal administration and comprising 5.6 mg cyclobenzaprine HCl in one or more dosage units, the cyclobenzaprine HCl being in the form of a eutectic comprising 75%±2% cyclobenzaprine HCl and 25%±2% β-mannitol, and the one or more dosage units further comprising a basifying agent.

In some embodiments, disclosed herein, is a composition for use in treating a subject believed to have fibromyalgia and its associated symptoms of pain, sleep disturbance and/or fatigue, the composition being suitable for transmucosal administration and comprising 65%±2% cyclobenzaprine HCl and 35%±2% δ-mannitol eutectic. In some embodiments, disclosed herein is a composition for use in treating a subject believed to have fibromyalgia and its associated symptoms of pain, sleep disturbance and/or fatigue, the composition being suitable for transmucosal administration and comprising a mixture of a 75%±2% cyclobenzaprine HCl and 25%±2% β-mannitol and a 65%±2% cyclobenzaprine HCl and 35%±2% δ-mannitol eutectic. In some embodiments, disclosed herein is a composition for use in treating a subject believed to have fibromyalgia and its associated symptoms of pain, sleep disturbance and/or fatigue, the composition being suitable for transmucosal administration and comprising a granule comprising an outer layer of a 65%±2% cyclobenzaprine HCl and 35%±2% δ-mannitol eutectic and an inner layer of β-mannitol.

In some embodiments, the associated symptoms are pain, disturbed sleep, and/or fatigue. In some embodiments, the associated symptom is pain. In some embodiments, the associated symptom is disturbed sleep. In some embodiments, the associated symptom is pain. In some embodiments, the associated symptom is fatigue.

### EXAMPLES

### Example 1. Study Design

A randomized, double-blind placebo-controlled study in patients with fibromyalgia was performed in 39 U.S. sites with a full sample size of N=503. Subjects were randomized 1:1 and placebo comparator arm (N=255) received a placebo once-daily at bedtime, while the experimental arm (N=248) received 5.6 mg cyclobenzaprine HCl one per day at bedtime in two dosage units by transmucosal administration, the cyclobenzaprine HCl being in the form of a eutectic comprising 75%±2% cyclobenzaprine HCl and 25%±2% mannitol, and each dosage unit further comprising a basifying agent. All subjects received one tablet of 2.8 mg cyclobenzaprine HCl, as above, or placebo for the first two weeks, which was increased to two tablets of 2.8 mg cyclobenzaprine HCl (5.6 mg total), as above, or placebo for the remaining 12 weeks. The primary endpoint of the study was 14 weeks in which 82.3% of the experimental arm and 83.5% of the placebo arm completed the study.

### Example 2. Primary and Secondary Endpoints

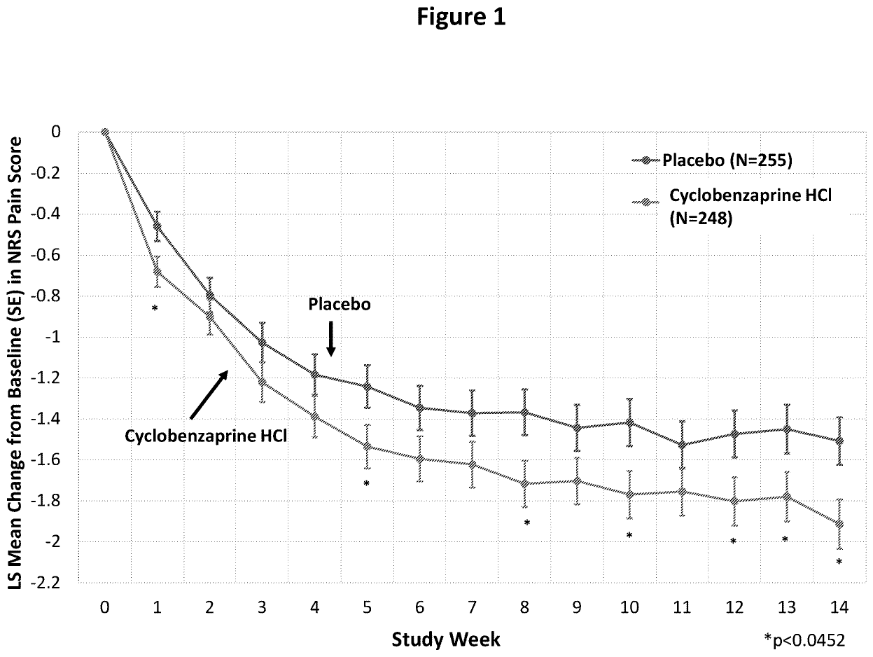

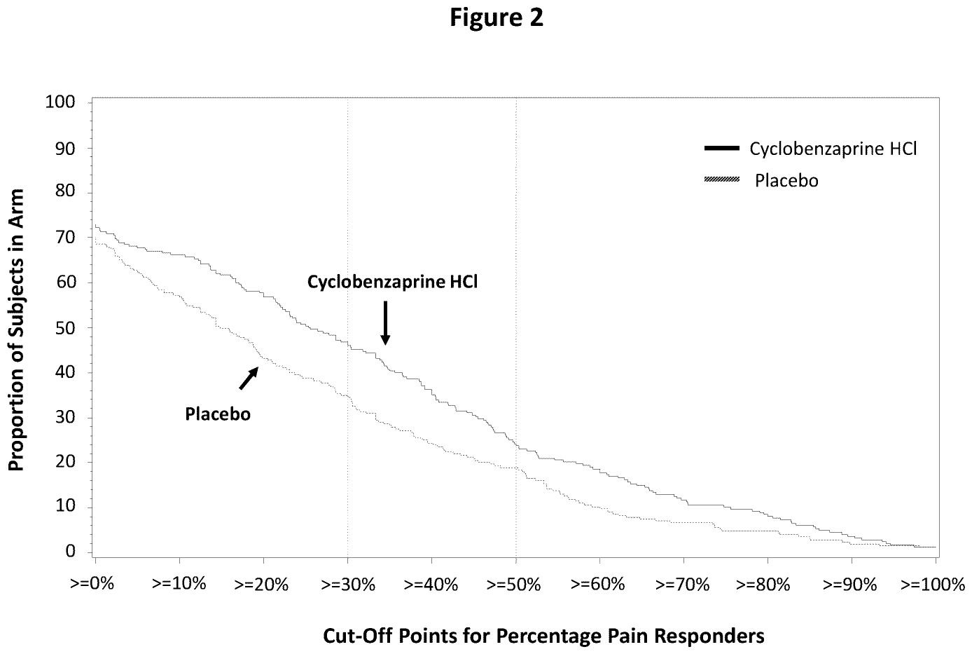

The primary outcome measure of mean pain score at Week 14 was measured by daily pain diary severity score change (cyclobenzaprine HCl versus placebo) from baseline in the weekly average using the numerical rating scale (NRS). Change from baseline in the weekly average of daily diary pain severity NRS scores for experimental arm (LS mean [SE]: −1.9 [0.12] units) versus placebo (−1.5 [0.12] units) was analyzed by mixed model repeated measured analysis with multiple imputation (MMRM with MI) (LS mean [SE] difference: −0.4 [0.16] units, p=0.010). There was improvement of pain in the experimental arm as compared to the placebo group at Week 14 and during the duration of the 14-week study (FIG. 1, Table 1, and Table 2). Daily diary pain was also analyzed using Continuous Responder Analysis (CRA), which measured the proportion of responders over a range cut-off points (e.g., 0% to 100%) for percentage pain responders (FIG. 2). Statistical analysis showed that a 30% responder analysis, with 46.8% in the experimental arm and 34.9% in the placebo arm had a 30 percent or greater reduction in pain (logistic regression; odds ratio [95% CI]:1.67 [1.16, 2.40]; p=0.006) (FIG. 2). A greater proportion of the experimental arm indicated improved reduction in pain as compared to the placebo group at all cut-off points up to >=95%.

TABLE 1
Primary Efficacy Endpoint

| Primary | | | | |
|---|---|---|---|---|
| Outcome | | | | |
| Measure | | Cyclobenzaprine | | |
| at | Placebo | HCl dosage unit | Treatment | P- |
| Week 14 | (N = 255) | (N = 248) | Difference | value |
| | LS Mean | LS Mean Change | Difference in | |
| | Change from | from Baseline | LS Mean Change | |
| | Baseline | (SE) | from Baseline | |
| | (SE) | | Between | |
| | | | Cyclobenzaprine | |
| | | | HCl dosage unit | |
| | | | and Placebo (SE) | |
| Daily Pain | −1.5 (0.12) | −1.9 (0.12) | −0.4 (0.16) | 0.010* |
| Diary, | | | | |
| NRS | | | | |

Statistical Method: Mixed Model Repeated Measures analysis with Multiple Imputation
*p < 0.0452 (requisite p-value hurdle for full study after Interim Analysis)
¹ Same primary endpoint for FDA approvals of Cymbalta ® and Lyrica ® in fibromyalgia
Abbreviations: LS = least squares; NRS = numeric rating scale; SE = standard error

TABLE 2
Results of Primary and Secondary Endpoints

| | Intent-to-Treat | |
|---|---|---|
| Outcome Measure at Week 14 | Analysis | P-value¹ |
| Primary Endpoint | | |
| Daily Pain Diary, NRS | Mean Change (Primary | 0.010* |
| | Analysis)² | |
| Daily Pain Diary, NRS | Proportion with ≥30% | 0.006^{#} (LR) |
| | Improvement in | |
| | Pain³ | |
| Secondary Endpoints | | |
| Non-specific | | |
| Patient Global Impression of | Proportion “Much | 0.058 |
| Change | Improved” or “Very | |
| | Much Improved” | |
| Fibromyalgia-Syndrome | | |
| Related | | |
| FIQ-R Symptom Domain | Mean Change | 0.007^{#} |
| FIQ-R Function Domain | Mean Change | 0.009^{#} |
| PROMIS Fatigue | Mean Change | 0.018^{#} |
| Daily Sleep Quality Diary, | Mean Change | <0.001^{#} |
| NRS | | |
| PROMIS Sleep Disturbance | Mean Change | <0.001^{#} |

FIQ-R = Fibromyalgia Impact Questionnaire-Revised;
PROMIS = Patient-Reported Outcomes Measurement Information System;
LR = Logistic Regression (missing data considered non-responders);
NRS = Numeric Rating Scale
*statistically significant at p < 0.0452
^{#}nominally significant at p < 0.0452
¹Analysis by Mixed Model Repeated Measures with Multiple Imputation unless otherwise indicated.
²Primary endpoint analysis for FDA approvals of Cymbalta ® and Lyrica ® in fibromyalgia.
³Primary endpoint for FDA approval of Savella ® in fibromyalgia.

Key secondary end points at Week 14 include (1) Patient Global Impression of Change (PGIC) (responder analysis); (2) Fibromyalgia Impact Questionnaire—Revised (FIQ-R) symptom domain score (mean change); (3) FIQ-R function domain score (mean change); (4) PROMIS Sleep Disturbance instrument T-score (mean change); (5) PROMIS Fatigue instrument T-score (mean change); and (6) daily diary NRS assessment of sleep quality (mean change) (Table 2). The responder analysis of PGIC trended toward a greater proportion of responders (rating of “very much improved” or “much improved” at Week 14) to cyclobenzaprine HCl dosage unit of 37.5% compared with placebo of 29.4%. The result did not achieve statistical significance (logistic regression; odds ratio [95% CI]:1.44 [0.99, 2.10]; p=0.058). PGIC is a general measure of patient self-assessed benefit that is not tied to any specific symptom of fibromyalgia.

The experimental arm showed nominal improvement of sleep (Table 2). The diary sleep quality ratings of cyclobenzaprine HCl dosage unit (−2.0 [0.12] units) compared to placebo (−1.5 [0.12] units) was nominally significant (LS mean difference: −0.6 [0.17] units; p<0.001). The PROMIS Sleep Disturbance instrument, the experimental arm was also nominally significant over the placebo arm on T-scores (LS mean difference: −2.9 [0.82] units; p<0.001). The effect sizes on the diary sleep ratings and PROMIS Sleep Disturbance instrument were 0.31 and 0.32, respectively. The experimental arm also showed nominal improvement over placebo on the PROMIS Fatigue instrument T-scores (−1.8 [0.76] units; p=0.018). The syndromal activity of cyclobenzaprine HCl dosage unit was studied by the Fibromyalgia Impact Questionnaire—Revised (FIQ-R). The experimental arm showed nominal improvement the over placebo arm in both the symptom domain (−4.3 [1.60] units; p=0.007) and function domain (−4.4 [1.69] units; p=0.009).

### Example 3. Safety Results

Cyclobenzaprine HCl dosage units of this disclosure were well-tolerated. In the experimental arm, administration site reactions were the most commonly reported adverse events, including tongue/mouth numbness, tongue/mouth pain and/or discomfort, taste impairment, and tongue/mouth tingling, which were higher in the experimental arm than placebo arm (Table 3). Tongue/mouth numbness or tingling and taste impairment were local effects temporally related to dose administration and transiently expressed (<60 minutes) in most occurrences. The only systemic treatment-emergent adverse events that occurred at a rate of 5.0% or greater in either arm was somnolence/sedation at 5.6% in the experimental arm, which was consistent with known side effects of marketed oral cyclobenzaprine. Adverse events resulted in premature study discontinuation in 8.9% of those who received cyclobenzaprine HCl dosage unit compared with 3.9% of placebo recipients. There were a total of 7 serious adverse events in the study, none of which were deemed related to the investigational product; 5 in placebo arm, and 2 in experimental arm. Of the 2 in the experimental arm, one was a motor vehicle accident with multiple bone fractures, and the other was a pneumonia secondary to an infection.

TABLE 3
Safety and Adverse Events

| Cyclobenzaprine | | | | | |
|---|---|---|---|---|---|
| HCl dosage unit | | Placebo | | Total | |
| (N = 248) | | (N = 255) | | (N = 503) | |
| N | % | N | % | N | % |

Administration Site Reactions

| | | | | | | |
|---|---|---|---|---|---|---|
| Tongue/mouth numbness | 43 | 17.3 | 2 | 0.8 | 45 | 8.9 |
| Tongue/mouth | 29 | 11.7 | 5 | 2.0 | 34 | 6.8 |
| pain/discomfort | | | | | | |
| Taste impairment | 16 | 6.5 | 1 | 0.4 | 17 | 3.4 |
| Tongue/mouth tingling | 14 | 5.6 | 1 | 0.4 | 15 | 3.0 |

Systemic Adverse Events

| | | | | | | |
|---|---|---|---|---|---|---|
| Somnolence/Sedation | 14 | 5.6 | 3 | 1.2 | 17 | 3.4 |

### Example 4. More Detailed Analysis of the Treatment& Emergent Adverse Events

A more detailed analysis of the adverse events is reported in Table 4. In the experimental arm, oral cavity adverse events were commonly reported treatment emergent adverse events (TEAE), including hypoaesthesia oral, paraesthesia oral, dysgeusia, glossodynia, and dry mouth, which were higher in the experimental arm than placebo arm. The incidence of oral TEAE was 40.7% in the cyclobenzaprine HCl dosage unit group and 9.0% in the placebo group. Oral hypoaesthesia, the most common adverse event, was never rated as severe but led to only one discontinuation. Sedation and fatigue were the only systemic treatment-emergent adverse events that occurred at a rate of 3.6% in the experimental arm. Adverse events resulted in premature study discontinuation in 8.9% of those who received cyclobenzaprine HCl dosage unit compared with 3.9 of placebo recipients.

TABLE 4
Treatment Emergent Adverse Events

| | Cyclobenzaprine | | | | | |
|---|---|---|---|---|---|---|
| System Organ Class & | HCl dosage unit | | Placebo | | Total | |
| Preferred Terms | N | % | N | % | N | % |

Oral Cavity Adverse Events

| | | | | | | |
|---|---|---|---|---|---|---|
| Hypoaesthesia oral | 43 | 17.3 | 1 | 0.4 | 44 | 8.7 |
| Paraesthesia oral | 14 | 5.6 | 1 | 0.4 | 15 | 3.0 |
| Dysgeusia | 13 | 5.2 | 1 | 0.4 | 14 | 2.8 |
| Glossodynia | 9 | 3.6 | 2 | 0.8 | 11 | 2.2 |
| Dry mouth | 8 | 3.2 | 7 | 2.7 | 15 | 30. |

Systemic Adverse Events

| | | | | | | |
|---|---|---|---|---|---|---|
| Sedation | 9 | 3.6 | 1 | 0.4 | 10 | 2.0 |
| Fatigue | 9 | 3.6 | 4 | 1.6 | 13 | 2.6 |

1.-34. (canceled)

35. A multiple-variable dose method for treating fibromyalgia and one or more of its associated symptoms of pain, sleep disturbance, or fatigue in a subject in need thereof, comprising:
the transmucosal administration of a total of 2.8 mg of cyclobenzaprine HCl in one or more first dosage units once daily to the subject for about two weeks; and
following the about two weeks of the once daily administration of the total of 2.8 mg of cyclobenzaprine HCl in the one or more first dosage units, the transmucosal administration of a total of 5.6 mg of cyclobenzaprine HCl in one or more second dosage units to the subject once daily for as long as needed,
wherein the cyclobenzaprine HCl in each of the one or more first dosage units and the one or more second dosage units are in the form of a cyclobenzaprine HCl-mannitol eutectic,
wherein the cyclobenzaprine HCl-mannitol eutectic is a 75%±2% cyclobenzaprine HCl by weight and 25%±2% β-mannitol by weight eutectic, and
wherein each of the one or more first dosage units and the one or more second dosage units further comprise a basifying agent.

36. The method for treating according to claim 35, wherein the one or more of the second dosage units that comprise the 5.6 mg cyclobenzaprine HCl total dose are two dosage units, each dosage unit comprising 2.8 mg of cyclobenzaprine HCl.

37. The method for treating according to claim 35, wherein each of the one or more first dosage units and the one or more second dosage units are administered at bedtime.

38. The method for treating according to claim 35, wherein said transmucosal administration comprises sublingual, buccal, intranasal or palatal administration.

39. The method for treating according to claim 38, wherein said transmucosal administration is sublingual administration.

40. The method for treating according to claim 39, wherein the one or more first dosage units and the one or more second dosage units are administered in a dosage form selected from a sublingual tablet, a sublingual film, a sublingual liquid, sublingual powder, or a sublingual spray solution.

41. The method for treating according to claim 35, wherein the basifying agent is selected from a group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate, trisodium citrate, borate, hydroxide, silicate, nitrate, dissolved ammonia, bicarbonate, and sulfide.

42. The method for treating according to claim 41, wherein the basifying agent is dipotassium hydrogen phosphate.

43. The method for treating according to claim 35, wherein the symptom associated with fibromyalgia is pain.

44. The method for treating according to claim 43, wherein the pain is reduced by 30% or greater in a population of the subjects who are administered the cyclobenzaprine HCl-mannitol eutectic as compared to a population of the subjects who are administered a placebo.

45. The method for treating according to claim 44, wherein the reduction in pain for each subject is measured by a daily diary pain numerical rating scale severity score change from the numerical rating scale severity score at the start of the administration of the cyclobenzaprine HCl-mannitol eutectic or the placebo.

46. The method for treating according to claim 35, wherein the symptom associated with fibromyalgia is sleep disturbance.

47. The method for treating according to claim 46, wherein the sleep disturbance on average is reduced or sleep quality on average improves in a population of the subjects who are administered the cyclobenzaprine HCl-mannitol eutectic as compared to a population of the subjects who are administered a placebo.

48. The method for treating according to claim 35, wherein the symptom associated with fibromyalgia is fatigue.

49. The method for treating according to claim 48, wherein the fatigue is on average reduced in a population of the subjects who are administered the cyclobenzaprine-HCl-mannitol eutectic as compared to a population of the subjects who are administered a placebo.

50. The method for treating according to claim 35, wherein the subject is human.
